(19) United States
(12) Patent Application Publication
(10) Pub. No.: US 2007/0189942 A1
(43) Pub. Date: Aug. 16, 2007

(54) Sterilizer For Medical Devices

(75) Inventor: Hwa-Jung RYU, Pusan (KR)

Correspondence Address: CLARK & ELBING LLP, 101 FEDERAL STREET, BOSTON, MA, 02110, US

(73) Assignee: SEJIN-TECH Co., LTD., Pusan (KR)

(21) Appl. No.: 11/565,895

(22) Filed: Dec. 1, 2006

(30) Foreign Application Priority Data
Feb. 14, 2006 (KR) 2006-0028312

(51) Int. Cl. A61L 2/18

(52) U.S. Cl. 422/300

(57) ABSTRACT

Disclosed is a sterilizer for medical devices. The sterilizer includes a circulation unit composed of a pump and a pipe for circulating sterilizing liquid. The sterilizing liquid is selected from the group consisting of oil including paraffin oil and liquidus sterilizing agent. A heating unit is installed at one side of the pipe of the circulation unit. The heating unit heats up the sterilizing liquid passing through inside of the pipe. A hanger unit is provided for holding an upper end of the medical device such that the medical device is placed in vertical direction thereof. The hanger unit includes a nozzle connected to the circulation unit so that the sterilizing liquid being circulated through the circulation unit is sprayed from the upper portion to the lower portion of the medical device to be flown along the inner and outer side of the medical device. The heated sterilizing liquid flows from the upper side to the lower side of the medical device to sterilize the medical device.

### CROSS-REFERENCE TO OTHER RELATED APPLICATIONS

This application claims the benefit of Korean Patent Application No. 2006-0028312, filed on Feb. 14, 2006, in the Korean Intellectual Property Office, the disclosure of which is incorporated herein in its entirety by reference.

### BACKGROUND OF THE INVENTION

1. Field of the Invention

The present invention relates to a sterilizer for medical devices, and more specifically to a sterilizer, in which a high-temperature sterilizing liquid is flown into the outer and inner surfaces of medical devices to remove bacteria stuck to the surfaces of the medical devices.

2. Background of the Related Art

Bacteria or virus stuck to the surfaces of used medical devices may cause a secondary infection to other patients. Thus, used medical devices must be sterilized before using them again. For this purpose, conventionally, the used medical devices have been boiled or exposed to high-temperature steam. In this way, if the medical devices are heated up, the bacteria can be removed from the surface thereof, but some of the bacteria may remain on the surface thereof. In addition, the conventional heat-up sterilizing method embraces a disadvantage of inconvenience in its use. Further, in case of utilizing chemicals such as alcohol, certain types of bacteria or virus may remain still active. In order to solve this problem, Korean Patent Application No. 2004-0022274 discloses a sterilizing apparatus and method. In this application, a used medical device is placed inside of a perforated container. The, the perforated container is dipped into a sterilization bath containing a paraffin flow-oil of high-temperature to thereby performing sterilization or disinfection. In this approach, sterilization is carried out within the perforated container dipped into the sterilization bath, and after sterilization, the perforated container is taken out of the sterilization bath to take out the sterilized medical devices. Thus, it is more convenient and effective, as compared with direct-boiling using a pot or the like. In the above-mentioned method, however, excessive oil comes to remain on the surface of devices, thereby requiring a subsequent oil-removing process. However, the oil cannot be easily removed.

### SUMMARY OF THE INVENTION

The present invention has been made in order to solve the above problems with conventional technologies, and it is an object of the invention to provide a sterilizer for medical devices, which can sterilize the medical devices more conveniently and easily remove oil or the like stuck to the surface thereof.

In order to accomplish the above object, according to one aspect of the invention, there is provided a sterilizer for medical devices, the sterilizer comprising: a circulation unit composed of a pump and a pipe for circulating sterilizing liquid, the sterilizing liquid being selected from the group consisting of oil including paraffin oil and liquidus sterilizing agent; a heating unit installed at one side of the pipe of the circulation unit for heating the sterilizing liquid passing through inside of the pipe; and a hanger unit for holding an upper end of the medical device such that the medical device is placed in vertical direction thereof, the hanger unit including a nozzle connected to the circulation unit so that the sterilizing liquid being circulated through the circulation unit is sprayed from the upper portion to the lower portion of the medical device to be flown along the inner and outer side of the medical device, wherein the heated sterilizing liquid flows from the upper side to the lower side of the medical device to sterilize the medical device.

Preferably, the circulation unit further includes a lower bath for collecting the sterilizing liquid dropping from the medical device after sterilizing the medical device.

Preferably, the lower bath is provided with a filter at the connection portion with the pump of the circulation unit so as to filter the sterilizing liquid.

Preferably, the hanger unit is configured to move upward and downward such that the medical device held in the hanger unit can be dipped in the sterilizing liquid collected in the lower bath.

Preferably, the lower bath includes a heating means for heating the sterilizing liquid collected in the lower bath.

Preferably, the sterilizer further comprises a high-pressure air supplier for supplying high-pressure air to the hanger unit such that the sterilizing liquid remained in the medical device is dropped to the lower side.

Preferably, the high-pressure air supplier is further provided with a heating device for heating the high-pressure air passing through a pipe, the heating device being installed around the pipe.

### BRIEF DESCRIPTION OF THE DRAWINGS

FIG. 1 is a perspective view of a sterilizer for medical devices according to an embodiment of the invention.

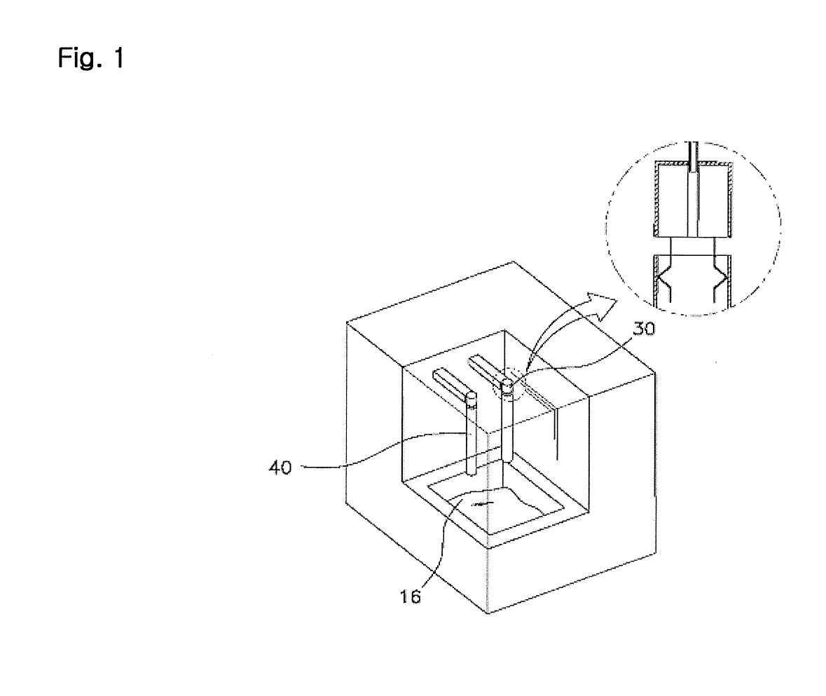

FIG. 2 illustrates a structure of the sterilizer of FIG. 1

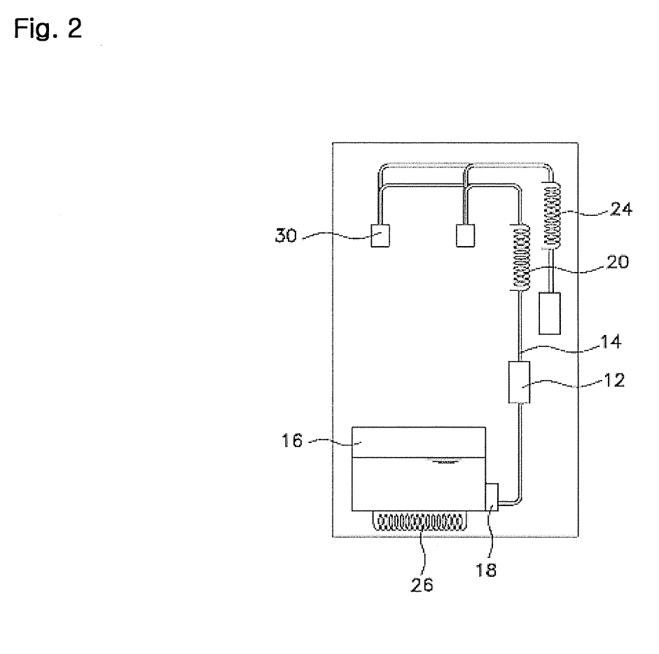

### DETAILED DESCRIPTION OF THE PREFERRED EMBODIMENT

Hereinafter, a sterilizer for medical devices according to an embodiment of the invention will be explained in detail, with reference to the accompanying drawings.

The sterilizer of this embodiment includes, in general, a circulation unit, a heating unit 20, and a hanger unit 30 and a high-pressure air supplier unit.

The circulation unit includes a pump 12 and a pipe 14 connected to the pump 12. For sterilization, the circulation unit circulates throughout the sterilizer various oils such as flowable paraffin oil, chemicals, and various sterilizing agents. The circulation unit is provided at its one side with a lower bath 16, into which sterilizing agents are dropped after sterilizing the medical devices. Installed at one side of the lower bath 16 is a filter 18, which screens out foreign matters from the sterilizing liquid contained in the lower bath 16. The filtered sterilizing liquid can be re-circulated by means of the pump 12. The lower bath 16 may be provided with a heating means 26 for the sterilizing liquid collected in the lower bath 16. In case where the lower bath 16 has a heating means 26, the sterilizing liquid can be maintained at a desired temperature to thereby improve its sterilizing effect and efficiency. That is, the sterilizing liquid passing through the pipe 14 is heated only by the heating unit 20, it may not be heated up to a desired temperature within a short period of time. However, if the sterilizing liquid heated-up in the lower bath is heated again by the heating unit 20, it can be maintained at a temperature required for sterilization.

Next, the heating unit 20 will be explained. The heating unit 20 is installed surrounding the pipe 14, through which the sterilizing liquid suctioned through the pump 12 flows and is heated up. The heating unit 20 may employ various equipment such as heating coil, gas-burner and the like. The heating unit heats up the sterilizing liquid up to about 80 to 140° C. The temperature may be adjusted appropriately, depending upon the types of medical devices to be sterilized.

Hereafter, the hanger unit 30 will be explained. The hanger 30 holds a medical device to be sterilizer. For this purpose, preferably the hanger 30 may take an appropriate structure according to the medical devices to be sterilized. That is, in case of a hand piece 40 or an endoscope having a through-hole formed inside thereof, the hanger unit 30 is structured such that one end of the medical device can be inserted into one end of the hanger 30. However, instead of having different shapes for different medical devices, the hanger unit 30 may have a clamp-type, which can grip various types of medical devices. The hanger unit 30 is provided with a duct and a nozzle, through which the sterilizing liquid being circulated by the circulation unit flows inside and outside of the medical devices. This embodiment illustrates one duct, but may include multiple ducts for flowing the sterilizing liquid inside and outside of the medical device through individual ducts. In addition, the hanger unit 30 is configured to move up and down. Thus, a medical device fixed to the hanger unit 30 can be partially or in its entirety dipped into the lower bath 16 and sterilized.

Next, the high-pressure air supplier unit is explained. The high-pressure air supplier unit is structured such that high-pressure air is sprayed through the duct and nozzle of the hanger unit 30. For this purpose, in this embodiment, high-pressure air is contained in a storage vessel 22, which is connected to the duct of the hanger unit 30 through an air hose. Instead of containing high-pressure air in the storage vessel 22, an air pump 12 may be provided for supplying air. In addition, instead of using the duct through which the sterilizing liquid flows, a separate air duct may be provided. Due to operation of the circulation unit, sterilizing liquid comes to stick to the surface of the medical device. Thus, the sterilizing liquid is to be removed for convenience of re-use. Therefore, after completion of the operation of the circulation unit, the high-pressure air supplier unit is operated to spray high-pressure air onto the medical device to remove the sterilizing liquid stuck thereto. In addition, a heating device 24 is further provided in the air hose of the high-pressure air supplier unit to heat the air. The heating device 24 is installed around the air hose using a heating coil. Instead of the heating coil, a gas burner or the like may be employed.

Hereafter, the operation of the present invention will be explained, with reference to the accompanying drawings.

As shown in the figures, the sterilizer for medical devices according to the invention includes a hanger unit 30 provided at its upper side and a lower bath 16 provided at its lower side. The lower bath 16 is opened to the upper side thereof. A heating means 26 is provided at the lower side of the lower bath 16. The heating means 26 is configured to heat up the lower bath 16. The lower bath 16 is connected at one lower side thereof with a circulation unit. Here, a filter 18 is installed between the circulation unit and the lower bath 16 such that foreign matters contained in the sterilizing liquid of the lower bath 16 can not be transferred to the circulation unit. The circulation unit is provided with a pump 12, which suctions the sterilizing liquid from the lower bath 16 and transfers it to the hanger unit 30. At this time, the pipe 14 installed between the pump 12 and the hanger unit 30 is provided with a heating unit 20 made of a heating coil to heat up again the sterilizing liquid. In addition, a storage vessel 22 is provided at one side to contain high-pressure air. The storage vessel 22 is connected with the hanger unit 30 through an air hose. With the above-constructed sterilizer for medical devices, a hand piece 40 used in dental offices can be fitted into the hanger unit 30 in vertical direction of the hand piece. With the hand piece 40 fitted into the hanger unit, the nozzle of the hanger unit comes to place above the hand piece 40. After placing the hand piece as above, the sterilizing liquid is heated up to 100° C. and then the pump 12 of the circulation unit is operated. Accordingly, the high-temperature sterilizing liquid is sprayed form the upper portion to the lower portion of the hand piece 40 to kill bacteria and virus while flowing along the inner through-hole and the outer surface of the hand piece. The above operation continues for 3 to 15 minutes and then the circulation unit stops its operation. Thereafter, the high-pressure air is sprayed to the lower side through the hanger unit 30 to remove the sterilizing liquid. At this time, in case of a hand piece, the sterilizing liquid is not completely removed, but remains on the surface. This is because in case of a hand piece, the sterilizing liquid partially remained in the hand piece facilitates a smooth operation of the hand piece. Besides the hand piece, in case of other medical devices, the high-pressure air is sprayed for a while sufficiently to completely remove the sterilizing liquid from the surface.

As described above, the sterilizer for medical devices according to the invention can easily remove bacteria, virus and the like stuck to the medical device to thereby prevent a secondary infection. In addition, the sterilizing liquid stuck to the surface can be removed to thereby enable to use conveniently.

Although the present invention has been described with reference to several exemplary embodiments, the description is illustrative of the invention and is not to be construed as limiting the invention. Various modifications, variations and replacements may occur to those skilled in the art, without departing from the spirit and scope of the invention as defined by the appended claims.

What is claimed is:

1. A sterilizer for medical devices, the sterilizer comprising:
a circulation unit composed of a pump and a pipe for circulating sterilizing liquid, the sterilizing liquid being selected from the group consisting of oil including paraffin oil and liquidus sterilizing agent;
a heating unit installed at one side of the pipe of the circulation unit for heating the sterilizing liquid passing through inside of the pipe; and
a hanger unit for holding an upper end of the medical device such that the medical device is placed in vertical direction thereof, the hanger unit including a nozzle connected to the circulation unit so that the sterilizing liquid being circulated through the circulation unit is sprayed from the upper portion to the lower portion of the medical device to be flown along the inner and outer side of the medical device, wherein the heated sterilizing liquid flows from the upper side to the lower side of the medical device to sterilize the medical device.

2. The sterilizer as claimed in claim 1, wherein the circulation unit further includes a lower bath for collecting the sterilizing liquid dropping from the medical device after sterilizing the medical device.

3. The sterilizer as claimed in claim 2, wherein the lower bath is provided with a filter at the connection portion with the pump of the circulation unit so as to filter the sterilizing liquid.

4. The sterilizer as claimed in claim 2 or 3, wherein the hanger unit is configured to move upward and downward such that the medical device held in the hanger unit can be dipped in the sterilizing liquid collected in the lower bath.

5. The sterilizer as claimed in claim 2 or 3, wherein the lower bath includes a heating means for heating the sterilizing liquid collected in the lower bath.

6. The sterilizer as claimed in claim 1, further comprising a high-pressure air supplier for supplying high-pressure air to the hanger unit such that the sterilizing liquid remained in the medical device is dropped to the lower side.

7. The sterilizer as claimed in claim 6, wherein the high-pressure air supplier is further provided with a heating device for heating the high-pressure air passing through a pipe, the heating device being installed around the pipe.
